(19) United States
(12) Patent Application Publication
(10) Pub. No.: US 2015/0209292 A1
(43) Pub. Date: Jul. 30, 2015

(54) CONTROLLED RELEASE FORMULATIONS AND PREPARATION METHOD THEREOF

(71) Applicant: TWi , Pharmaceuticals, Inc., Taipei (TW)

(72) Inventors: Chaur-Ming Jan, Taoyuan County (TW); Shao-Ming Lee, New Taipei City (TW); Chen-Yi Chang, Taoyuan County (TW)

(21) Appl. No.: 14/606,569

(22) Filed: Jan. 27, 2015

Related U.S. Application Data
(60) Provisional application No. 61/931,701, filed on Jan. 27, 2014.

(51) Int. Cl. A61K 9/28; A61K 31/4458; A61K 9/20

(52) U.S. Cl. CPC A61K 9/2846; A61K 9/2077; A61K 31/4458; A61K 9/2866

(57) ABSTRACT

Provided is a controlled release formulation and a preparation process thereof. Also provided is a method for treating Attention-Deficit Disorder (ADD) or Attention-Deficit Hyperactivity Disorder (ADHD).

### CROSS-REFERENCES TO RELATED APPLICATIONS

Not Applicable.

### BACKGROUND OF THE INVENTION

1. Field of the Invention

The present invention relates to a controlled release formulation for less frequent, preferably once daily administration, and a preparation method thereof. The present invention also relates to the use of the controlled release formulation in the treatment of Attention-Deficit Disorder (ADD) and Attention-Deficit Hyperactivity Disorder (ADHD).

2. Descriptions of the Related Art

It is known that a drug must be made available in appropriate concentration at its site of action within the body to produce its pharmacological effects, but if the drug concentration is beyond a certain range, undesired pharmaceutical effects, i.e., side effects, may generate. Thus, to maintain drug concentration within the body is a key issue in the design of a drug formulation.

Methylphenidate is a central nervous system stimulant used to treat ADD and ADHD, available commercially as, e.g., RITALIN SR®, CONCERTA®, METADATE® CD capsules and METEADATE® ER Tablets. The beneficial results seen by clinicians in the treatment of ADD and ADHD have resulted in widespread use of methylphenidate in more than two million patients annually. However, the use of methylphenidate may generate several side effects including anorexia, weight loss, insomnia, dizziness and dysphoria, and thus, controlling the release of methylphenidate to maintain its concentration within the body in a certain range to prevent the generation of these side effects is important.

Several dosage forms have been developed for controlling the release of methylphenidate. For example, U.S. Pat. No. 6,930,129 discloses an osmotic dosage form for delivering methylphenidate releasing drug at an ascending release rate over an extended time period. The process for manufacturing this dosage form requires a laser step to make an orifice on the semipermeable membrane for releasing methylphenidate, which makes the process complicated and costly and is adverse to the drug production in the plant scale accordingly. Therefore, there remains a need to provide a method that is more economical and convenient in production of a methylphenidate formulation compared to the conventional process while controlling the drug release at a desired rate and maintaining therapeutic drug effect over a prolonged therapy period.

The present invention thus provides a controlled release formulation to satisfy the above requirements. By controlling the release of a drug at a predetermined rate in the present invention, the drug can be administered in a single dose and provide the needed delivery rate so that a satisfactory balance of desired and undesired pharmacological effects over a prolonged therapy period can be maintained.

### SUMMARY OF THE INVENTION

The primary objective of this invention is to provide a controlled release formulation comprising:

(A) an outer compressed coat layer comprising
(i) an immediate release granule comprising a first active ingredient and a pharmaceutically acceptable excipient; and
(ii) a controlled release granule comprising a second active ingredient and a controlled release agent; and

(B) an inter layer comprising
(i) a core tablet comprising a third active ingredient and a controlled release agent; and
(ii) an optional controlled release film coating the core tablet.

Another objective of this invention is to provide a process of preparing a controlled release formulation, comprising:

(A) making an outer coat layer mixture, comprising the steps of:
(a) making an immediate release granule
   i. weighing and de-lumping a therapeutically effective amount of a first active ingredient;
   ii. mixing the first active ingredient and a pharmaceutically acceptable excipient to form a first mixture;
   iii. granulating said first mixture into a first granule by a shear mixer, and drying the first granule to obtain the immediate release granule;
   iv. optionally sieving the immediate release granule;
(b) making a controlled release granule
   i. weighing and de-lumping a therapeutically effective amount of a second active ingredient;
   ii. mixing the second active ingredient, a controlled release agent, and a pharmaceutically acceptable excipient to form a second mixture;
   iii. granulating said second mixture into a second granule by a shear mixer and drying the second granule to obtain the controlled release granule;
   iv. optionally slugging and sieving the controlled release granule; and
(c) blending and lubricating the immediate release granule and the controlled release granule to form the outer coat layer mixture; and

(B) making an inter layer, comprising the steps of:
i. weighing and de-lumping a therapeutically effective amount of a third active ingredient;
ii. mixing the third active ingredient, a controlled release agent, and a pharmaceutically acceptable excipient to form a third mixture;
iii. granulating said third mixture into a third granule by a shear mixer, and drying the third granule;
iv. optionally sieving the third granule;
v. lubricating and compressing the third granule into a core tablet;
vi. optionally coating a controlled release film onto the core tablet; and

(C) compressing the inter layer and the outer coat layer mixture simultaneously, allowing the inter layer to be compressed into the outer coat layer mixture to form the controlled release formulation, wherein the outer coat layer mixture forms into an outer compressed coat layer due to being compressed.

Still another objective of this invention is to provide a method for treating Attention-Deficit Disorder (ADD) or Attention-Deficit Hyperactivity Disorder (ADHD) in a patient, comprising administering the controlled release formulation of the present invention to the patient.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 is a diagram showing the structure of one embodiment of the controlled release formulation of the present invention;

FIG. 2 is a diagram showing the structure of another embodiment of the controlled release formulation of the present invention;

FIG. 3 is an exemplary flow chart showing the steps for preparing the controlled release formulation of the present invention;

FIGS. 4(A) to 4(C) show the dissolution profiles of the controlled release formulations A and B of the present invention, measured by the United States Pharmacopeia (USP) Apparatus II (Paddle) at 50 rpm in 500 ml of pH 6.8 PBS or 0.1N HCl at 37° C. with UV at 270 nm;

FIGS. 5(A) and 5(B) show the average plasma concentration-time profiles of methylphenidate with the Linear ordinate after subjects received the reference listed drug (Concerta®) and the controlled release formulation A of the present invention under a fasted or a fed condition, respectively;

FIGS. 6(A) and 6(B) show the average plasma concentration-time profiles of methylphenidate with the Linear ordinate after subjects received the reference listed drug (Concerta®) and the controlled release formulation B of the present invention under a fasted or a fed condition, respectively;

FIG. 7 shows the dissolution profiles of the controlled release granules G1 to G4, measured by the USP Apparatus II (Paddle) at 50 rpm in 500 ml of 0.1N HCl at 37° C. with UV at 270 nm;

FIGS. 8(A) and 8(B) show the dissolution profiles of the inter layers I1 and I2, measured by the USP Apparatus II (Paddle) at 50 rpm in 500 ml of 0.1N HCl or pH 6.8 PBS at 37° C. with UV at 270 nm; and

FIGS. 9(A) and 9(B) show the dissolution profiles of the inter layers I3, I4 and I5, measured by the USP Apparatus II (Paddle) at 50 rpm in 500 ml of 0.1N HCl or pH 6.8 PBS at 37° C. with UV at 270 nm.

### DETAILED DESCRIPTION OF THE INVENTION

### Term Definition

The term “Granule,” as used herein, shall be understood to encompass bead, spheroid, particulate, particle, pellet, microcapsule, microtablet, or other similar pharmaceutically acceptable configuration that can deliver a drug.

The term “Immediate Release” or “IR,” as used herein, means that a drug (e.g., methylphenidate) is released in a conventional or non-modified way, which is preferably greater than or equal to about 80% of the drug released within 0.5 hours of dissolution.

The term “Controlled Release” or “CR” and “Sustained Release” or “SR,” as used herein, refers to the gradual release of a drug at a predetermined rate other than an immediate release manner over a period of time.

The term “effective amount,” as used herein, refers to an amount that alleviates or reduces one or more symptoms of a disease.

The term “Cmax,” as used herein, refers to the maximum observed plasma concentration, calculated as the mean of the individual maximum blood plasma concentrations.

The term “average plasma concentration,” as used herein, refers to the arithmetic mean blood plasma concentration.

The term “Tmax,” as used herein, refers to the time at which the peak (maximum) observed blood plasma drug concentration for each individual participating in the bioavailability study.

The term “AUC0-∞” or “AUCinf,” as used herein, refers to the mean area under the plasma/serum/blood concentration-time curve extrapolated to infinity. It is calculated as the arithmetic mean of the area under the plasma concentration-time curve from time 0 extrapolated to infinity, calculated for each individual participating in the bioavailability study.

The term “AUC0-t,” as used herein, refers to the area under the plasma/serum/blood concentration-time curve from time zero to time t, where “t” is the last sampling time point with measurable concentration for individual formulation.

The term “partial AUC0-T1” or “pAUC0-T1,” as used herein, refers to the area under the plasma/serum/blood concentration-time curve from time zero to time T1, where “T1” is the predetermined first sampling time point.

The term “partial AUCT1-T2” or “pAUCT1-T2,” as used herein, refers to the area under the plasma/serum/blood concentration-time curve from time T1 to time T2, where “T1” and “T2” are the predetermined first and second sampling time points, respectively.

The term “partial AUCT2-T3” or “pAUCT2-T3,” as used herein, refers to the area under the plasma/serum/blood concentration-time curve from time T2 to time T3, where “T2” and “T3” are the predetermined second and third sampling time points, respectively.

In addition, unless otherwise stated herein, the terms “a (an)”, “the” or the like used in this specification (especially in the Claims hereinafter) shall be understood to encompass both the singular form and the plural form.

As stated above, to overcome the problems existing in conventional formulations, the present invention provides a controlled release formulation with a “tablet-in-tablet” or “press-coating” configuration. Based on this configuration and the ratios of components in the present invention, the controlled release formulation provides desired release rate and profile of methylphenidate. Specifically, the present invention provides a controlled release formulation comprising:

(A) an outer compressed coat layer comprising
(i) an immediate release granule comprising a first active ingredient and a pharmaceutically acceptable excipient; and
(ii) a controlled release granule comprising a second active ingredient and a controlled release agent; and

(B) an inter layer comprising
(i) a core tablet comprising a third active ingredient and a controlled release agent; and
(ii) an optional controlled release film coating the core tablet.

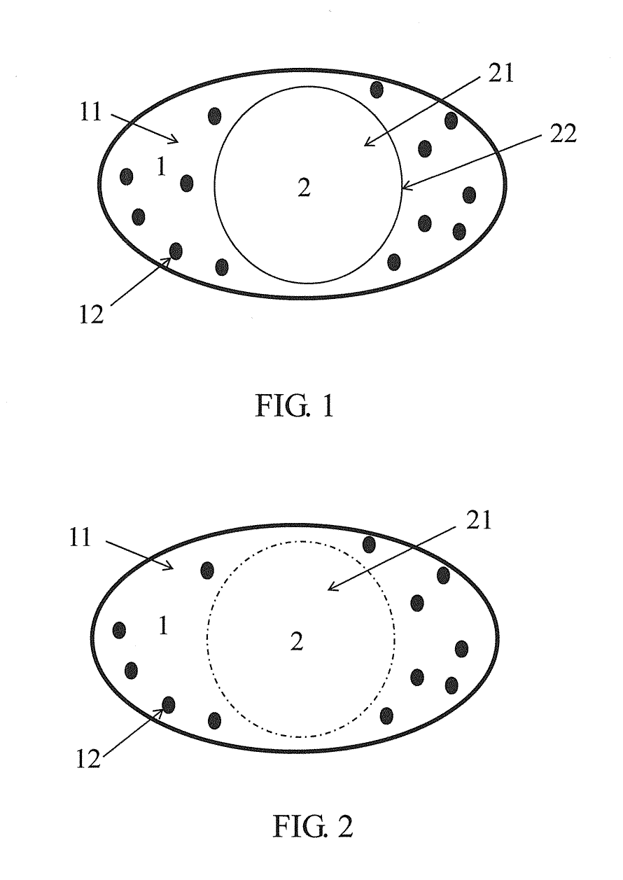

Referring to FIG. 1, showing one embodiment of the formulation of the present invention (the components are not drawn to scale), the structure of the controlled release formulation of the present invention comprises an outer compressed coat layer 1, which includes an immediate release granule 11 comprising a first active ingredient, and a controlled release granule 12 comprising a second active ingredient; and an inter layer 2, which includes a core tablet 21 comprising a third active ingredient and an optional controlled release film 22. This “tablet-in-tablet” configuration can provide a multiphasic release profile.

There is no particular limit on the first, second and third active ingredients in the present invention. These three active ingredients can be the same or different from each other, or only two of them are the same, depending on the practical requirement.

In one embodiment, the first, second and third active ingredients are different. Specifically, the controlled release formulation delivers the desired pharmacokinetic profile by releasing the active ingredients at different rates: the outer compressed coat layer 1 that releases the first active ingredient from the immediate release granule 11 in a non-modified way after administration, and releases the second active ingredient from the controlled release granule 12 following the release of the first active ingredient; and the third active ingredient is released from the inter layer 2 in a relatively slow and controlled way.

In another embodiment, the first, second and third active ingredients are the same; preferably the active ingredient is methylphenidate or its pharmaceutically acceptable salts. Specifically, the controlled release formulation delivers a desired pharmacokinetic profile by releasing methylphenidate at different rates: (1) the first portion of methylphenidate (i.e., the first active ingredient) is released from the immediate release granules in a non-modified way after dosing that provides a rapid onset; (2) the second portion of methylphenidate (i.e., the second active ingredient) is slowly released from the controlled release granules in the outer coat that maintains the plasma profile in the therapeutically effective level; (3) the third portion of methylphenidate (i.e., the third active ingredient) is released from the inter layer that provides a sustained release profile and achieves Tmax from about 5 to 12 hours after dosing.

For many drugs, it is beneficial that the plasma drug concentration initially ascends for a short period of time as drug release begins and then remains substantially constant over an extended time period as drug release continues at a constant rate. This substantially constant plasma drug concentration correlates with substantially constant drug effectiveness over a prolonged therapy period. The controlled release formulation of the subject invention has the above advantages. The pharmacological effect of the drug in the controlled release formulation can be rapidly generated right after the administration due to immediate release of the outer compressed coat layer 1, and then the drug continues to exert its effect due to the sustained release of the inter layer 2.

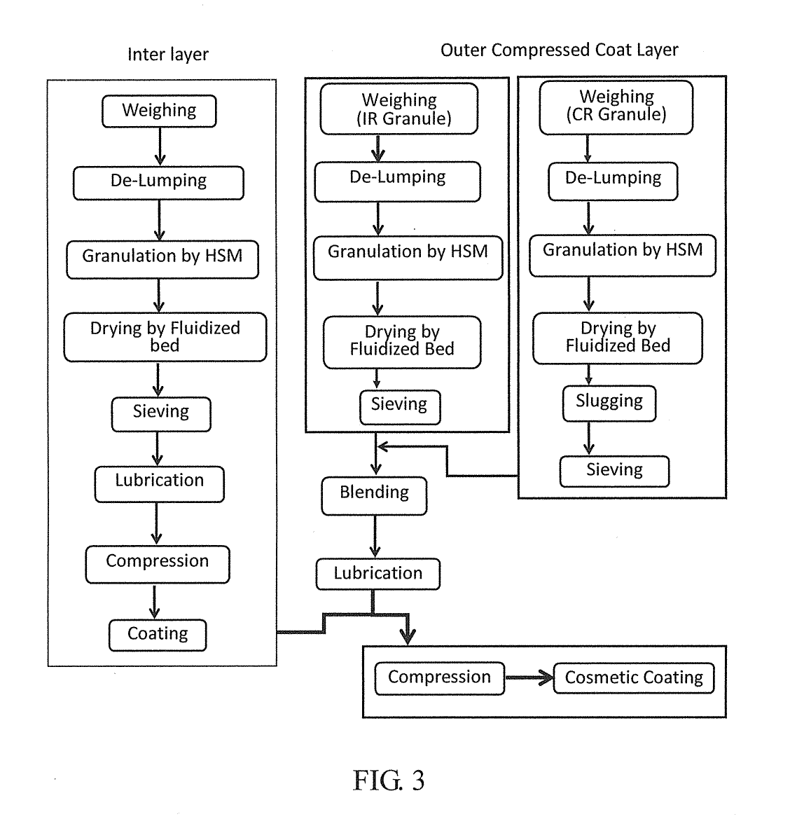

In one embodiment of the present invention, the configuration of the controlled release formulation may contain no controlled release film 22, i.e., the controlled release film 22 coating the core tablet 21 can be removed from the structure of the controlled release formulation as shown in FIG. 2. In this embodiment, the desired controlled release effect can be achieved by adjusting the ingredients and ratios in the core tablet 21.

“Methylphenidate salts” include derivatives of methylphenidate, wherein methylphenidate is modified by making acidic salts thereof, i.e., pharmaceutically acceptable salts. Examples of pharmaceutically acceptable salts include, but are not limited to, salts of mineral or organic acids, for example, hydrochloric, hydrobromic, hydrofluoric, sulfuric, phosphoric, acetic, trifluoroacetic, benzoic, citric, maleic, succinic or methanesulfonic salts. The hydrochloride salt is particularly preferred. Exemplary methylphenidate salts include methylphenidate benzoate, methylphenidate citrate, methylphenidate glutarate, methylphenidate hydrobromide, methylphenidate hydrochloride, methylphenidate hydrogen phosphate, methylphenidate dihydrogen phosphate, methylphenidate lactate, methylphenidate mandelate, methylphenidate maleate, methylphenidate mesylate, methylphenidate oxalate, methylphenidate sulfate, or a hydrate or solvate of the foregoing salts.

Methylphenidate or a pharmaceutically acceptable salt thereof used in the present invention may be in crystalline, amorphous, polymorphic, anhydrous or hydrate form, or a combination of the forms thereof.

The controlled release agent in the core tablet and controlled release granule can be any materials that commonly known in the art and may control the release of a drug at a predetermined rate. Examples of the controlled release agent include hydrophilic polymers, water-swellable polymers, water-insoluble polymers, pH-dependent polymers, hydrophobic materials, or mixtures thereof.

Examples of hydrophilic and/or water-swellable polymers include, but are not limited to, cellulosic polymers, including hydroxyalkyl celluloses and carboxyalkyl celluloses, such as hydroxypropyl methylcellulose (HPMC), hydroxypropylcellulose (HPC), hydroxyethylcellulose (HEC), polyethylene oxide (PEO), methylcellulose (MC), carboxymethylcellulose (CMC), powdered cellulose such as microcrystalline cellulose, cellulose acetate, salts thereof, and combinations thereof alginates, gums, including heteropolysaccharide gums and homopolysaccharide gums, such as xanthan, tragacanth, pectin, acacia, karaya, agar, guar, hydroxypropyl guar, veegum, carrageenan, locust bean gum, gellan gum, and derivatives thereof; acrylic resins, including polymers and copolymers of acrylic acid, methacrylic acid, methyl acrylate and methyl methacrylate and cross-linked polyacrylic acid derivatives such as Carbomers (e.g., CARBOPOL®, including CARBOPOL® 71G NF, available in various molecular weight grades from Noveon, Inc., Cincinnati, Ohio); polyvinyl acetate (e.g., KOLLIDON®SR); polyvinyl pyrrolidone and its derivatives such as crospovidone; and polyvinyl alcohol. Preferred hydrophilic and water-swellable polymers include the cellulosic polymers, especially HPMC. Persons skilled in the art will understand that different grades of HPMC can be used in the formulation according to the present invention. Examples include Methocel® K4M, Methocel® E5, Methocel® E50, Methocel® E4M, Methocel® K15M, Methocel® K100M, and Methocel® K100LV.

Examples of water-insoluble polymers that may be used include, but are not limited to, ethyl cellulose, methacrylic acid derivatives such as ammoniomethacrylate copolymer (EUDRAGIT® RL or EUDRAGIT® RS), methacrylic acid ester neutral copolymer (EUDRAGIT® NE30D), Kollicoat® SR30D (containing 27 wt % polyvinyl acetate and 2.5 wt % polyvinyl pyrrolidone) and the like and mixtures thereof. Examples of the hydrophobic materials that may be used includes, but are not limited to, waxes, carnauba wax, vegetable wax, fruit wax, microcrystalline wax, bees wax, hydrocarbon wax, paraffin wax, cetyl esters wax, nonionic emulsifying wax, anionic emulsifying wax, candelilla wax, stearyl alcohol, cetyl alcohol, cetostearyl alcohol, lauryl alcohol, myristyl alcohol, a hydrogenated vegetable oil, a hydrogenated castor oil, a fatty acid, a fatty acid ester, or mixtures thereof.

Examples of pH-dependent polymers include hydroxypropyl methylcellulose phthalate, cellulose acetate phthalate, carboxymethyl ethylcellulose, methyl methacrylate-methacrylic acid copolymer (Eudragit® L100 or Eudragit® S100), methacrylic acid-ethyl acrylate copolymer (Eudragit® L100-55 or Eudragit® L30D-55), methacrylic acid-methyl acrylate-methyl methacrylate copolymer (Eudragit® FS30D), hydroxypropyl cellulose acetate succinate, polyvinyl acetate phthalate and shellac etc.

The controlled release film coating the core tablet in the controlled release formulation of the present invention comprises a film-forming polymer, and the film-forming polymer can be, for example, pH-dependent polymers, pH-independent polymers, water-insoluble polymers, hydrophilic polymers, water-swellable polymers, or any combinations thereof. Preferably, the controlled release film comprises at least one pH-dependent polymer and at least one water-insoluble polymer. Examples of the pH-dependent polymer and the water-insoluble polymer are as those described above. In one embodiment of the present invention, the controlled release film comprises methacrylic acid copolymers as the pH-dependent polymer and ethyl cellulose as the water-insoluble polymer. Examples of methacrylic acid copolymers include Eudragit® L100, Eudragit® L100-55, Eudragit® S-100, Kollicoat® MAE 30DP, and Kollicoat® MAE 100P. Examples of ethyl cellulose include Ethocel® products, such as EC N-100, Aqualon® products, Surelease® dispersions, Aquacoat® ECD aqueous dispersion, etc.

The controlled release film also preferably contains plasticizers. Plasticizers that may be used include any of those known to those skilled in the art, including but not limited to, acetyl tributyl citrate, triacetin, acetylated monoglyceride, rape oil, olive oil, sesame oil, coconut oil, poloxamer, acetyl triethyl citrate, glycerin, sorbitol, diethyl oxalate, diethyl malate, diethyl fumarate, dibutyl succinate, diethyl malonate, dioctyl phthalate, dibutyl phthalate, dibutyl sebacate, triethyl citrate (TEC), tributyl citrate, glycerol tributyrate, polyethylene glycol, and any combinations thereof. The preferred plasticizer is triethyl citrate (TEC). The concentration of the plasticizer in the controlled release film may be, for example, about 0.5 wt % to about 5.0 wt %, preferably about 1.0 wt % to about 4.0 wt %, on the basis of the total weight of the controlled release film.

The controlled release film may also include an anti-sticking agent such as those selected from talc, colloidal silica dioxide, magnesium stearate, magnesium silicate, glyceryl monostearates, calcium stearate, stearic acid, or any combinations thereof. The preferred anti-sticking agent is talc. The concentration of the anti-sticking agent in the controlled release film may be, for example, about 20.0 wt % to about 60.0 wt %, preferably about 30.0 wt % to about 55.0 wt %, on the basis of the total weight of the controlled release film.

The controlled release formulation of the present invention may further comprise other pharmaceutically acceptable excipients. “Pharmaceutically acceptable excipients” refer to excipients that may have different functions in the formulation or are normally employed in the oral formulations, and after administration to or upon a subject, do not cause undesirable physiological effects. The excipient in a pharmaceutical composition must be “acceptable” also in the sense that it is compatible with the active ingredient. Pharmaceutically acceptable excipients can be selected by those skilled in the art using conventional criteria.

Pharmaceutically acceptable excipients include, but are not limited to, diluents, granulating aids, colourants, flavourants, surfactants, pH adjusters, lubricants, glidants, plasticizers, binders, fillers, extenders, humectants, disintegrants, wetting agents, etc. Examples of the excipients used in the invention include, but are not limited to, cellulose, microcrystalline, starch, corn starch, lactose, sucrose, glucose, mannitol, silicic acid, citric acid, crospovidone, sodium chloride, cetyl alcohol, glycerol monostearate, kaolin, bentonite clay, talc, calcium stearate, magnesium stearate, solid polyethylene glycols, etc.

In a bioequivalent test, the controlled release formulation of the present invention, when used for delivering methylphenidate salts, preferably exhibits a ratio of a geometric mean of logarithmic transformed AUC0-∞ of the controlled release formulation to a geometric mean of logarithmic transformed AUC0-∞ of the reference drug (Concerta®) of about 0.80 to about 1.20; a ratio of a geometric mean of logarithmic transformed AUC0-t of the controlled release formulation to a geometric mean of logarithmic transformed AUC0-t of the reference drug (Concerta®) of about 0.80 to about 1.20; a ratio of a geometric mean of logarithmic transformed pAUC0-T1 of the controlled release formulation to a geometric mean of logarithmic transformed pAUC0-T1 of the reference drug (Concerta®) of about 0.80 to about 1.20; a ratio of a geometric mean of logarithmic transformed pAUCT1-T2 of the controlled release formulation to a geometric mean of logarithmic transformed pAUCT1-T2 of the reference drug (Concerta®) of about 0.80 to about 1.20; a ratio of a geometric mean of logarithmic transformed pAUCT2-T3 of the controlled release formulation to a geometric mean of logarithmic transformed pAUCT2-T3 of the reference drug (Concerta®) of about 0.80 to about 1.20; or a ratio of a geometric mean of logarithmic transformed Cmax of the controlled release formulation to a geometric mean of logarithmic transformed Cmax of the reference drug (Concerta®) of about 0.80 to about 1.20.

Besides, the controlled release formulation preferably exhibits at least one of the following pharmacokinetic parameters: (i) a maximum plasma concentration Cmax of methylphenidate of about 7 to 30 ng/ml; (ii) an area under the concentration time curve AUC0-t or AUC0-∞ of methylphenidate of about 130 to 220 ng·hr/ml; and (iii) a first peak plasma Tmax1 of about 1 hour, and/or a second peak plasma Tmax2 of about 6.5 hours under a fasted or a fed condition after oral administration to a patient.

In a dissolution test, the controlled release formulation preferably has an in vitro dissolution rate when measured by the United States Pharmacopeia (USP) Apparatus II (Paddle) at 50 rpm in 500 ml of pH 6.8 PBS at 37° C. with UV at 270 nm, between 10% and 30%, preferably 20% and 30%, methylphenidate released after 1 hour; between 25% and 55%, preferably 25% and 45%, methylphenidate released after 2 hours; between 55% and 85%, preferably 60% and 80%, methylphenidate released after 64 hours; and not less than 90% methylphenidate released after 12 hours, by weight.

The controlled release formulation of the present invention also preferably has an in vitro dissolution rate when measured by the USP Apparatus II (Paddle) at 50 rpm in 500 ml of 0.1N HCl at 37° C. with UV at 270 nm, between 10% and 30%, preferably 20% and 30%, methylphenidate released after 1 hour; between 20% and 50%, preferably 20% and 40%, methylphenidate released after 2 hours; between 35% and 60%, preferably 40% and 55%, methylphenidate released after 4 hours; and between 65% and 90%, preferably not less than 70%, methylphenidate released after 8 hours, by weight.

The present invention also provides a controlled release formulation, comprising:

(A) an outer compressed coat layer comprising
(i) an immediate release granule comprising about 0.5% to about 20.0%, preferably about 3.0% to about 15.0%, by weight of a first active ingredient, on the basis of the total weight of the immediate release granule, and a pharmaceutically acceptable excipient; and
(ii) a controlled release granule comprising about 0.5% to about 30.0%, preferably about 5.0% to about 20.0%, by weight of a second active ingredient and about 0.5% to about 95.0%, preferably about 30.0% to about 90.0%, by weight of a controlled release agent, on the basis of the total weight of the controlled release granule; and

(B) an inter layer comprising
(i) a core tablet comprising about 20.0% to about 50.0%, preferably about 30.0% to about 40.0%, by weight of a third active ingredient and about 10.0% to about 50.0%, preferably about 15.0% to about 40.0%, by weight of a controlled release agent, on the basis of the total weight of the core tablet; and
(ii) an optionally controlled release film coating the core tablet, comprising about 20.0% to about 99.5%, preferably about 30.0% to about 95.0%, by weight of a film-forming polymer, on the basis of the total weight of the controlled release film.

Preferably, the weight ratio of the immediate release granule to the controlled release granule in the outer compressed coat layer ranges from 10/1 to 1/4, more preferably, from 2/1 to 1/2.

The types of the first, second and third active ingredients, the controlled release agent and the film-forming polymer in the controlled release formulation, as well as the pharmacokinetic profiles and bioequivalence of the controlled release formulation are as those described above.

The present invention further provides a process of preparing a controlled release formulation, comprising:

(A) making an outer coat layer mixture, comprising the steps of:
(a) making an immediate release granule
   i. weighing and de-lumping a therapeutically effective amount of a first active ingredient;
   ii. mixing the first active ingredient and a pharmaceutically acceptable excipient to form a first mixture;
   iii. granulating said first mixture into a first granule by a shear mixer, preferably a high shear mixer, and drying the first granule by, for example, fluidized bed or an oven, to obtain the immediate release granule;
   iv. optionally sieving the immediate release granule;
(b) making a controlled release granule
   i. weighing and de-lumping a therapeutically effective amount of a second active ingredient;
   ii. mixing the second active ingredient, a controlled release agent, and a pharmaceutically acceptable excipient to form a second mixture;
   iii. granulating said second mixture into a second granule by a shear mixer, preferably a high shear mixer, and drying the second granule by, for example, fluidized bed or an oven, to obtain the controlled release granule;
   iv. optionally slugging and sieving the controlled release granule; and
(c) blending and lubricating the immediate release granule and the controlled release granule to form the outer coat layer mixture; and

(B) making an inter layer, comprising the steps of:
i. weighing and de-lumping a therapeutically effective amount of a third active ingredient;
ii. mixing the third active ingredient, a controlled release agent, and a pharmaceutically acceptable excipient to form a third mixture;
iii. granulating said third mixture into a third granule by a shear mixer, preferably a high shear mixer, and drying the third granule by, for example, fluidized bed or an oven;
iv. optionally sieving the third granule,
v. lubricating and compressing the third granule into a core tablet;
vi. optionally coating a controlled release film onto the core tablet; and

(C) compressing the inter layer and the outer coat layer mixture simultaneously, allowing the inter layer to be compressed into the outer coat layer mixture to form the controlled release formulation, wherein the outer coat layer mixture forms into an outer compressed coat layer due to being compressed.

In the process of the present invention, preferably, a 30 mesh sieve is used to conduct the steps of de-lumping the first, second and third active ingredients, and a 20 mesh sieve is used to perform the steps of sieving the immediate release granule, controlled-release granule, and the third granules. This sieving step is performed to obtain a preferred particle size range of the granules, which is beneficial to the control of drug release.

Preferably, the first mixture can be formed by pre-mixing the first active ingredient with a pharmaceutically acceptable excipient other than a binder for a period of time (e.g., three to five minutes), and then adding a binder thereinto; and the second mixture can be formed by pre-mixing the second active ingredient with the controlled release agent for a period of time (e.g., three to five minutes), and then adding a binder thereinto.

Besides, in the step (c), the immediate release granule and controlled release granule are lubricated by a lubricant, which is used to facilitate manufacturing of the formulation. Examples of the lubricant that can be used herein including, but are not limited to, talc, glyceryl monostearates, calcium stearate, magnesium stearate, stearic acid, glyceryl behenate, and polyethylene glycol, or any combinations thereof. Preferably, talc is used in this step.

In step (B), a lubricant is also used to lubricate the third granule. Examples of the lubricant that can be used in this step include, but are not limited to, talc, glyceryl monostearates, calcium stearate, magnesium stearate, stearic acid, glyceryl behenate, and polyethylene glycol, or any combinations thereof. Preferably, magnesium stearate is used in this step.

As stated on the above, the process for manufacturing methylphenidate osmotic dosage form disclosed in U.S. Pat. No. 6,930,129 is complicated and costly in production due to a necessary laser step. The preparation process of the present invention, however, does not require this laser step, and thus is more economical and convenient in production compared to the conventional process.

The present invention are also directed to a method for treating Attention-Deficit Disorder (ADD) or Attention-Deficit Hyperactivity Disorder (ADHD) in a patient, comprising administering the controlled release formulation of the present invention to the patient in need.

Hereinafter, the present invention will be further illustrated with reference to the following examples. However, these examples are only provided for illustrate purpose, but not to limit the scope of the present invention.

### Preparation Example

### Preparation of Methylphenidate Controlled Release Formulation

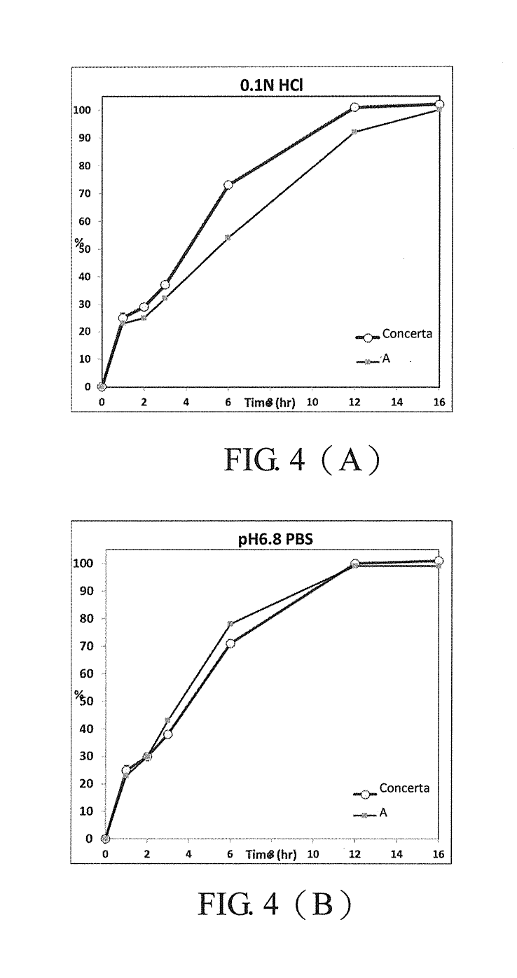

Two methylphenidate controlled release formulations A and B were prepared according to the flow chart in FIG. 3, and the components and ratios (as well as the preferred concentrations) are listed in Table 1. The preparation method was as follows:
(A) making an outer coat layer mixture, comprising the steps of:
   (a) making an immediate release granule
      i. weighing methylphenidate HCl and de-lumping it with Mesh #30 from Comil®;
      ii. pre-mixing methylphenidate HCl, Lactose 200M and MCC PH101 for 3 minutes, and then adding PVP K30 binder thereinto to form a first mixture;
      iii. granulating said first mixture into a first granule by a high shear mixer;
      iv. placing the wet first granule into a chamber and drying it by fluidized bed until the maximum granule moisture (L.O.D.) was not more than 3.0%, so as to obtain the immediate release granule;
      v. sieving the dried immediate release granule through Mesh #20 from Comil®;
   (b) making a controlled release granule
      i. weighing methylphenidate HCl and de-lumping it with Mesh #30 from Comil®;
      ii. pre-mixing methylphenidate HCl and HPMC K4M CR for 3 minutes, and then adding PVP K30 binder thereinto to form a second mixture;
      iii. granulating said second mixture into a second granule by a high shear mixer;
      iv. placing the wet second granule into a chamber and drying it by fluidized bed until the maximum granule moisture (L.O.D.) was not more than 3.0%, so as to obtain the controlled release granule;
      v. sieving the dried controlled release granule through Mesh #20 from Comil®;
      vi. slugging the controlled release granule by Roller Compactor and Pulverize from Comil®;
   (c) blending the immediate release granule with the controlled release granule for 5 minutes by V-blender, and lubricating the granules with talc for 5 minutes using V-blender to form the outer coat layer mixture; and
(B) making an inter layer, comprising the steps of:
   i. weighing methylphenidate HCl and de-lumping it with Mesh #30 from Comil®;
   ii. pre-mixing methylphenidate HCl, HPMC K100M CR and Lactose 200M to form a third mixture;
   iii. granulating said third mixture into a third granule by a high shear mixer;
   iv. placing the wet third granule into a chamber and drying it by fluidized bed until the maximum granule moisture (L.O.D.) was not more than 3.0%;
   v. sieving the dried third granule through Mesh #20 from Comil®;
   vi. lubricating the third granule with magnesium stearate for 5 minutes by V-blender;
   vii. adding Syloid 244FP to the third granule;
   viii. compressing the third granule into the inter layer (core tablet) by rotary press;
   ix. coating a controlled release film onto the inter layer;
(C) compressing the inter layer and the outer coat layer mixture simultaneously by rotary press, allowing the inter layer to be compressed into the outer coat layer mixture to form the controlled release formulation, wherein the outer coat layer mixture forms into an outer compressed coat layer due to being compressed; and
(D) optionally applying a cosmetic coating on the controlled release formulation.

TABLE 1

| Formulation | | | | | | | | |
|---|---|---|---|---|---|---|---|---|
| (Methylphenidate HCl, | | | | | | | Preferred | More preferred |
| 54 mg) | | | A | | B | | range | range |
| | | Ingredient | mg | % | mg | % | % | % |
| Inter layer | Core tablet | Methylphenidate | 42.12 | 35.1 | 42.12 | 35.1 | 20.0~50.0 | 30.0~40.0 |
| | | HCl | | | | | | |
| | | Lactose 200M | 51.96 | 43.3 | 52.38 | 43.7 | 20.0~60.0 | 30.0~50.0 |
| | | HPMC K100M | 24.00 | 20.0 | 24.00 | 20.0 | 10.0~50.0 | 15.0~40.0 |
| | | CR | | | | | | |
| | | Syloid ® 244FP | 0.75 | 0.625 | 0.75 | 0.63 | 0~2.0 | 0.5~1.0 |
| | | Mg Stearate | 0.75 | 0.625 | 0.75 | 0.63 | 0~2.0 | 0.5~1.0 |
| | | lake pigment | 0.42 | 0.35 | — | — | — | — |
| | | Subtotal | 120.0 | 100.0 | 120.0 | 100.0 | — | — |
| | Controlled | Eudragit L100 | 1.922 | 13 | 3.12 | 15.12 | 5.0~50.0 | 10.0~30.0 |
| | release film | EC N-100 | 2.363 | 16 | 3.12 | 15.12 | 5.0~60.0 | 10.0~40.0 |
| | | Klucel EF | 2.846 | 19.1 | 3.75 | 18.17 | 10.0~40.0 | 10.0~30.0 |
| | | TEC | 0.426 | 2.9 | 0.60 | 2.91 | 0.5~5.0 | 1.0~4.0 |
| | | Talc | 7.235 | 49.0 | 10.05 | 48.69 | 20.0~60.0 | 30.0~55.0 |
| | | Subtotal | 14.792 | 100.0 | 20.7 | 100.0 | — | — |
| Outer compressed | IR granule | Methylphenidate | 8.91 | 4.03 | 7.92 | 5.3 | 0.5~20.0 | 3.0~15.0 |
| coat layer | | HCl | | | | | | |
| | | Lactose 200M | 30.00 | 13.57 | 30.00 | 20.0 | 0~80.0 | 10.0~60.0 |
| | | MCC PH101 | 177.66 | 80.4 | 109.08 | 72.7 | 0~85.0 | 20.0~85.0 |
| | | PVP K30 | 2.23 | 1.01 | 1.50 | 1.0 | 0~5.0 | 0.0~3.0 |
| | | Syloid ® 244FP | 0.60 | 0.27 | — | — | — | — |
| | | Talc | 1.60 | 0.72 | 1.50 | 1.0 | 0~5.0 | 0.5~3.0 |
| | | Subtotal | 221.00 | 100.0 | 150.0 | 100.0 | — | — |
| | CR granule | Methylphenidate | 2.97 | 9.9 | 3.96 | 13.2 | 0.5~30.0 | 5.0~20.0 |
| | | HPMC K4M | 26.53 | 88.43 | 25.74 | 85.8 | 0.5~95.0 | 30.0~90.0 |
| | | CR | | | | | | |
| | | PVP K30 | 0.30 | 1.0 | 0.3 | 1.0 | 0~5.0 | 0.0~3.0 |
| | | Mg Stearate | 0.20 | 0.67 | — | — | — | — |
| | | Subtotal | 30.0 | 100.0 | 30.0 | 100.0 | — | — |

### Example 1

### Dissolution Assay for Methylphenidate Controlled Release Formulation

Dissolution

A dissolution profile is a plot of the cumulative amount of active agent released as a function of time. A dissolution profile can be measured utilizing the USP <724> Drug Release and the USP <711> Dissolution. A profile is characterized by the test conditions selected such as, for example, apparatus type, shaft speed, temperature, volume, and pH of the dissolution medium. More than one dissolution profile can be measured. For example, a first dissolution profile can be measured at a pH level approximating that of stomach, and a second dissolution profile can be measured at a pH level approximating that of one point in the intestine or several pH levels approximating multiple points in the intestine.

In this example, dissolution was performed in accordance with the USP Apparatus II (Paddle). Hydrochloric acid (0.1N) or pH 6.8 PBS was used as the dissolution medium. Samples were taken at suitable time intervals and analyzed for methylphenidate content by means of high-pressure liquid chromatography (HPLC).

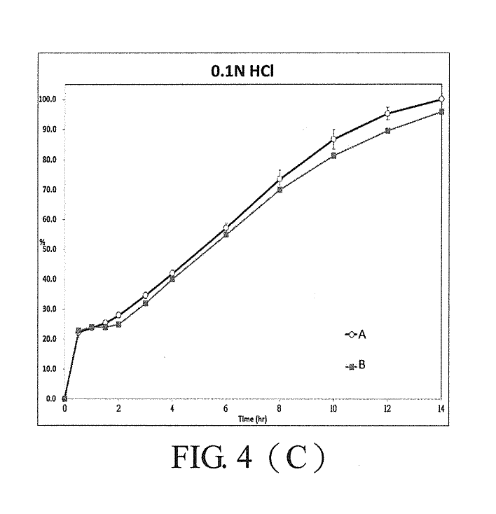

Table 2 summarizes the raw data of the dissolution of the formulations A and B of the present invention, as well as the reference listed drug, CONCERTA®, under different conditions, and FIGS. 4(A) to 4(C) show the dissolution profiles of the formulations A and B and CONCERTA®.

TABLE 2

| | Methylphenidate HCl (54 mg) | | | | | |
|---|---|---|---|---|---|---|
| | 0.1N HCl | | pH 6.8 PBS | | 0.1N HCl | |
| Time (hr) | Concerta ® | A | Concerta ® | A | A | B |
| 0 | 0.0 | 0.0 | 0.0 | 0.0 | 0 | 0 |
| 1 | 25 | 23 | 25 | 23 | 24 | 24 |
| 2 | 29 | 25 | 30 | 30 | 28 | 25 |
| 3 | 37 | 32 | 38 | 43 | 35 | 32 |
| 4 | — | — | — | — | 42 | 40 |
| 6 | 73 | 54 | 71 | 78 | 57 | 55 |
| 8 | — | — | — | — | 73.6 | 70 |
| 12 | 101 | 92 | 100 | 99 | 95 | 89 |
| 16 | 102 | 100 | 101 | 99 | — | — |

Dissolution method: USP Apparatus II (Paddle) * 50 rpm/medium * 500 ml with sinker/37° C. with UV at 270 nm

As shown in Table 2 and FIGS. 4(A) to 4(C), the dissolution of methylphenidate in the controlled release formulation of the present invention initially ascended for about 0.5 hour at a higher rate, mostly from the immediate release granule, and about 25% of methylphenidate was released after one hour. The dissolution, mainly from the controlled release granule and core tablet, then remained substantially constant over an extended time period for at least 12 hours.

### Example 2

### Pharmacokinetic Assay for Methylphenidate Controlled Release Formulation

Pharmacokinetic Study

Bioequivalent of methylphenidate composition to a reference drug can be determined by an in vivo bioequivalent study to determine a pharmacokinetic parameter for the methylphenidate composition. Specifically, bioequivalence can be determined by an in vivo bioequivalence study comparing a pharmacokinetic parameter for the two compositions. The test composition and reference drug are administered and plasma levels of the active agent are measured over time. Pharmacokinetic parameters characterizing rate and extent of active agent absorption are evaluated statistically.

An open-randomized, two-way, crossover study of the formulations A and B versus CONCERTA® (reference listed drug) was performed in normal healthy subjects using the strength of 54 mg. This study addresses the bioequivalence of methylphenidate from the formulations A and B under relevant clinical conditions.

Under U.S. FDA guidelines, generally, two products (e.g., an inventive composition and CONCERTA®) are bioequivalent if a ratio of geometric mean of logarithmic transformed AUC0-∞ or AUC0-t and Cmax for the two products are about 0.80 to about 1.25.

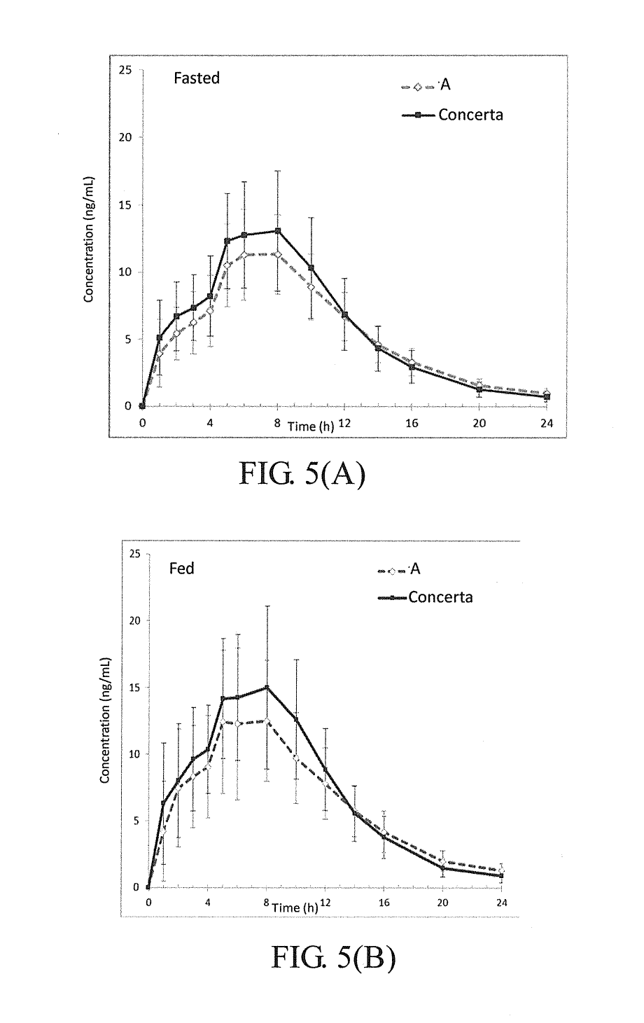

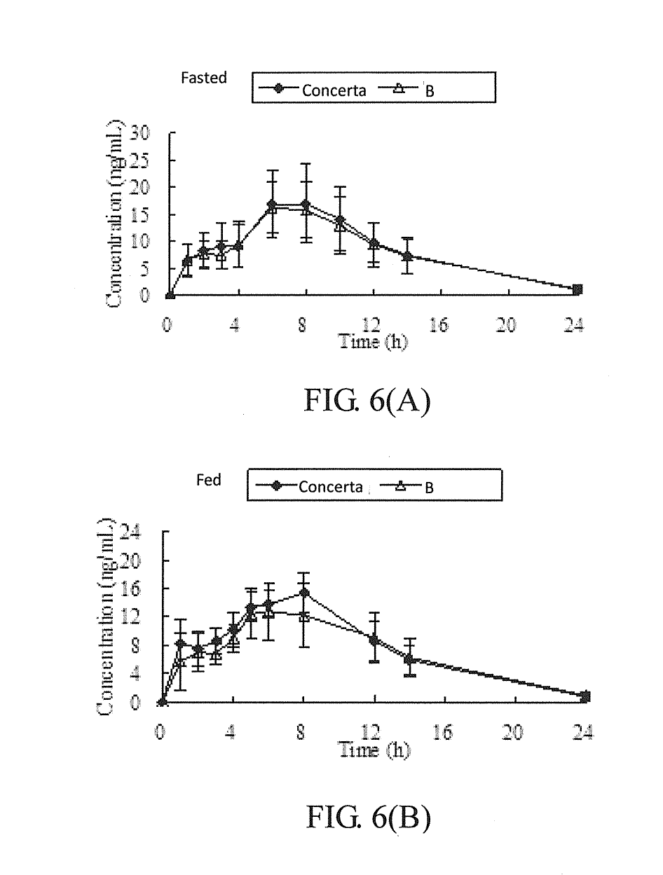

Tables 3 to 6 summarize the mean pharmacokinetic parameters of methylphenidate after subjects received CONCERTA® and the formulations A and B, respectively. FIGS. 5(A) to 6(B) show the average plasma concentration-time profiles of methylphenidate after subjects received CONCERTA® and the formulations A and B.

These results show that the formulations A and B of the present invention provide an immediate loading dose of methylphenidate, extends the duration of methylphenidate release and maintains an adequate plasma concentration for at least 24 hours. Besides, the formulation A and B are bioequivalent to CONCERTA®.

TABLE 3
Under Fasted State

| Formulation | | Concerta ® | A | Concerta ® | B |
|---|---|---|---|---|---|
| AUC_{0-∞} | Mean | 148.1 | 137.9 | 207.9 | 200.9 |
| | CV % | 32.7 | 26.2 | 41.3 | 36.9 |
| AUC₀₋ₜ | Mean | 143.6 | 131.4 | 202.4 | 193.6 |
| | CV % | 32.2 | 26.2 | 41.2 | 36.6 |
| Cₘₐₓ | Mean | 13.9 | 11.8 | 17.5 | 16.9 |
| | CV % | 31.0 | 27.8 | 38.2 | 29.5 |
| Tₘₐₓ₂ (hr) | Mean | 6.9 | 6.7 | 7.4 | 6.6 |
| | CV % | 20.5 | 20.6 | 13.1 | 14.9 |

*Concentration unit for AUC: ng · hr/mL; for Cₘₐₓ: ng/mL. CV means coefficient of variation.

TABLE 4
Under Fed State

| Formulation | | Concerta ® | A | Concerta ® | B |
|---|---|---|---|---|---|
| AUC_{0-∞} | Mean | 178.1 | 164.0 | 182.7 | 169.9 |
| | CV % | 33.8 | 34.4 | 19.9 | 25.2 |
| AUC₀₋ₜ | Mean | 172.8 | 154.7 | 179.1 | 165.3 |
| | CV % | 33.3 | 34.7 | 19.1 | 24.3 |
| Cₘₐₓ | Mean | 15.8 | 13.4 | 15.5 | 14.0 |
| | CV % | 37.8 | 41.3 | 16.7 | 24.0 |
| Tₘₐₓ₂ (hr) | Mean | 6.2 | 6.7 | 7.4 | 6.8 |
| | CV % | 33.5 | 22.3 | 16.1 | 46.7 |

TABLE 5
Under Fasted State

| | Formulation | | | |
|---|---|---|---|---|
| | Formulation A/Concerta ® | | Formulation B/Concerta ® | |
| | Geometric Mean | CV | Geometric Mean | CV |
| Parameters | (Ratio) | % | (Ratio) | % |
| AUC_{0-∞} | 0.978 | 10 | 0.976 | 10 |
| pAUC₀₋₃ | 0.861 | 16 | 0.947 | 23 |
| pAUC₃₋₇ | 0.875 | 15 | 0.981 | 16 |
| pAUC₇₋₁₂ | 0.906 | 16 | 0.944 | 9 |
| AUC₀₋ₜ | 0.961 | 10 | 0.967 | 10 |
| Cₘₐₓ | 0.869 | 19 | 0.989 | 16 |

TABLE 6
Under Fed State

| | Formulation | | | |
|---|---|---|---|---|
| | Formulation A/Concerta ® | | Formulation B/Concerta ® | |
| | Geometric Mean | CV | Geometric Mean | CV |
| Parameters | (Ratio) | % | (Ratio) | % |
| AUC_{0-∞} | 0.920 | 6.6 | 0.915 | 18.4 |
| pAUC₀₋₄ | 0.843 | 21.4 | 0.799 | 37.1 |
| pAUC₄₋₈ | 0.845 | 16.0 | 0.868 | 16.5 |
| pAUC₈₋₁₂ | 0.808 | 18.4 | 0.840 | 34.1 |
| AUC₀₋ₜ | 0.892 | 7.4 | 0.909 | 18.5 |
| Cₘₐₓ | 0.840 | 16.0 | 0.894 | 16.3 |

### Example 3

### The HPMC Level Effect on Drug Release of Controlled Release Granule

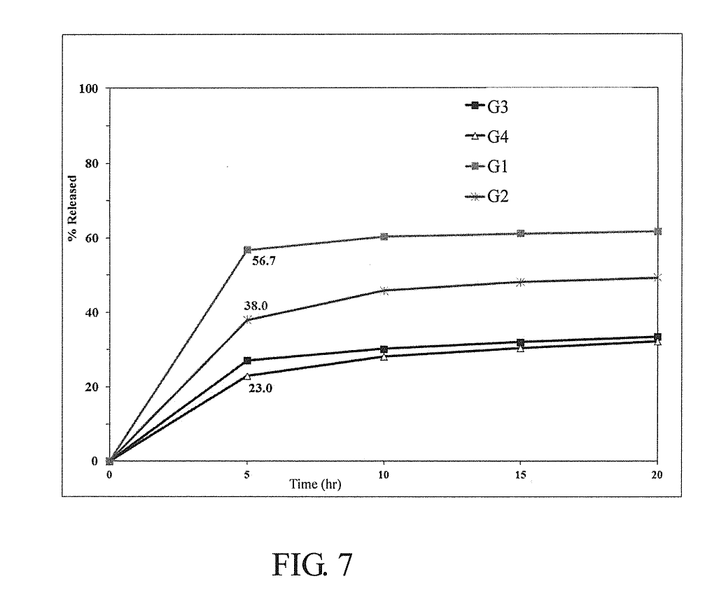

Controlled release granules G1 (containing 45.0% of a controlled release agent, HPMC), G2 (containing 60.0% of HPMC), G3 (containing 90.0% of HPMC), and G4 (containing 94.2% of HPMC) were prepared according to the following Table 7, and the HPMC level effect on the release rate of methylphenidate in controlled release granules was observed. The dissolution test was conducted with the same method as that described in Example 1. The results are shown in FIG. 7.

TABLE 7

| | Controlled Release Granule | | | | | | | |
|---|---|---|---|---|---|---|---|---|
| | G1 | | G2 | | G3 | | G4 | |
| Ingredient | mg | % | mg | % | mg | % | mg | % |
| Methyl- | 2.7 | 5.4 | 2.7 | 5.4 | 2.7 | 9.0 | 2.7 | 4.7 |
| phenidate HCl | | | | | | | | |
| HPMC K4M | 22.5 | 45.0 | 22.5 | 60.0 | 27.0 | 90.0 | 54.0 | 94.2 |
| CR | | | | | | | | |
| MCC PH 101 | 23.55 | 47.1 | 16.05 | 32.1 | — | — | — | — |
| PVP K30 | 1.25 | 2.5 | 1.25 | 1.5 | 0.30 | 1.0 | 0.6 | 1.0 |
| Total | 50.0 | 100 | 50.0 | 100 | 30.0 | 100 | 57.3 | 100 |

FIG. 7 shows that increasing the ratio of HPMC slowed the dissolution rate and better controlled the release of methylphenidate, and thus the therapy period of the drug can be extended.

### Example 4

### The HPMC Level Effect on Drug Release of Inter Layers

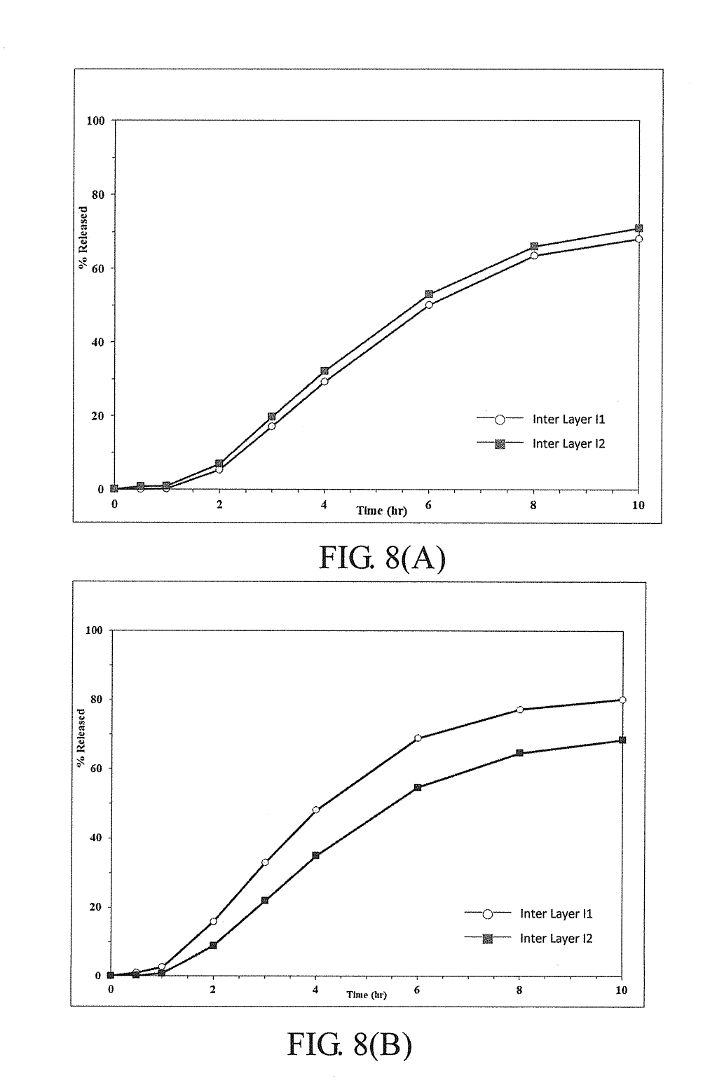

Inter layers I1 (containing 20.0% of HPMC) and I2 (containing 25.0% of HPMC) were prepared according to the following Table 8, and the HPMC level effect on the release rate of methylphenidate was observed. The dissolution test was conducted with the same method as that described in Example 1. The results are shown in FIGS. 8(A) and 8(B).

TABLE 8

| Inter Layer | | | I1 | | I2 | |
|---|---|---|---|---|---|---|
| | | Ingredient | mg | % | mg | % |
| Inter layer | Core tablet | Methylphenidate HCl | 42.12 | 35.1 | 42.12 | 35.1 |
| | | Lactose 200M | 52.38 | 43.7 | 46.38 | 38.7 |
| | | HPMC K100M CR | 24.0 | 20.0 | 30.00 | 25.0 |
| | | Syloid 244FP | 0.75 | 0.63 | 0.75 | 0.63 |
| | | Mg Stearate | 0.75 | 0.63 | 0.75 | 0.63 |
| | | Subtotal | 120.0 | 100.0 | 120.0 | 100.0 |
| | Sealing | Klucel EF | 1.50 | 40.0 | 1.50 | 40.0% |
| | layer | Talc | 2.25 | 60.0 | 2.25 | 60.0% |
| | | Subtotal | 3.75 | 100.0 | 3.75 | 100.0 |
| | Controlled release | Eudragit ® L100 | — | — | — | — |
| | film | Eudragit ® L100-55 | 2.0 | 9.66 | 2.0 | 9.66 |
| | | EC N-100 | 4.0 | 19.32 | 4.0 | 19.32 |
| | | Klucel EF | 4.05 | 19.57 | 4.05 | 19.57 |
| | | TEC | 0.60 | 2.90 | 0.60 | 2.90 |
| | | Talc | 10.05 | 48.55 | 10.05 | 48.55 |
| | | Subtotal | 20.7 | 100.0 | 20.7 | 100.0 |

In FIG. 8(A), the dissolution profiles of the inter layers I1 and I2 are not significantly different from each other, and the controlled release effect of HPMC was unobvious. It is possible that the pH-dependent polymer, Eudragit® L100-55, in the controlled release film was not dissolved in the 0.1N HCl solution (pH=1), and thus the function of HPMC was restricted thereby. However, as can be seen from FIG. 8(B), when in the PBS solution with pH 6.8, under which Eudragit® L100-55 is dissolved, it is obvious that the increase of HPMC (the inter layer I2) decreased the dissolution rate and better controlled the release of methylphenidate, and thus the therapy period of the drug can be extended.

### Example 5

### The Effect of the Polymer Ratio in Controlled Release Film on Drug Release

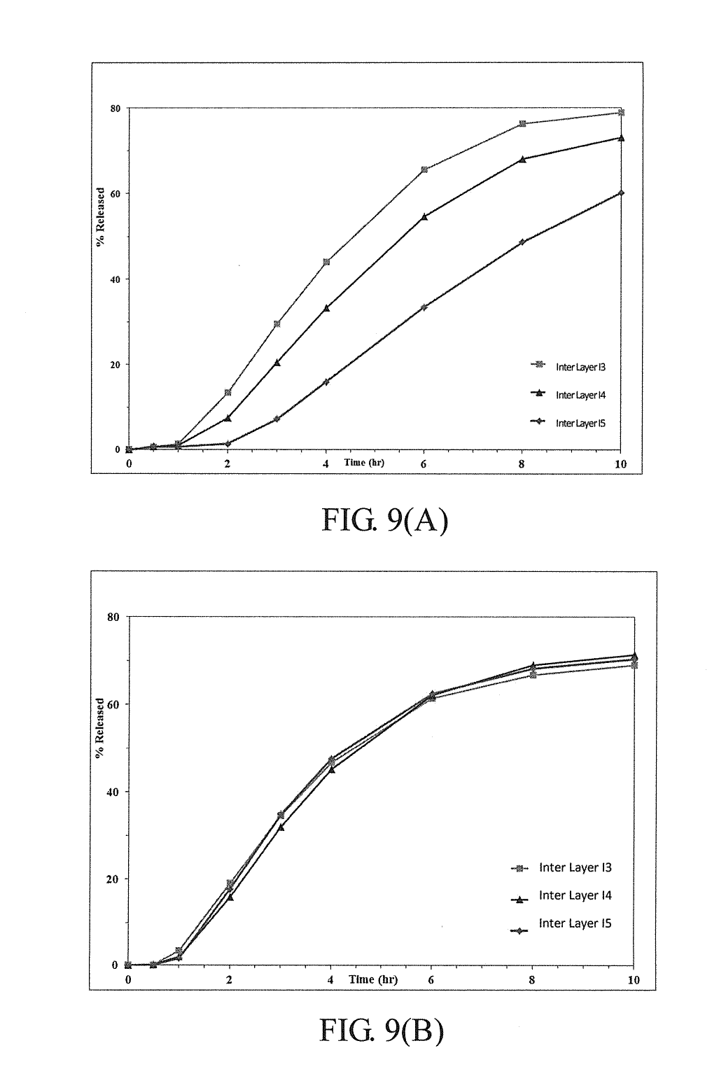

Inter layers I3, I4 and I5 were prepared according to the following Table 9, and the effect of the polymer ratio in the controlled release film on the release rate of methylphenidate was observed. The dissolution test was conducted with the same method as that described in Example 1. The results are shown in FIGS. 9(A) and 9(B).

TABLE 9

| Inter Layer | | | I3 | | I4 | | I5 | |
|---|---|---|---|---|---|---|---|---|
| | | Ingredient | mg | % | mg | % | mg | % |
| Inter layer | Core tablet | Methylphenidate HCl | 42.12 | 35.1 | 42.12 | 35.1 | 42.12 | 35.1 |
| | | Lactose 200M | 52.38 | 43.7 | 52.38 | 43.7 | 52.38 | 43.7 |
| | | HPMC K100M CR | 24.0 | 20.0 | 24.0 | 20.0 | 24.0 | 20.0 |
| | | Syloid 244FP | 0.75 | 0.63 | 0.75 | 0.63 | 0.75 | 0.63 |
| | | Mg Stearate | 0.75 | 0.63 | 0.75 | 0.63 | 0.75 | 0.63 |
| | | Subtotal | 120.0 | 100.0 | 120.0 | 100.0 | 120.0 | 100.0 |
| | Controlled release film | Eudragit ® L100 | — | — | 2.08 | 10.08 | 3.12 | 15.12 |
| | | Eudragit ® L100-55 | 2.08 | 10.08 | — | — | — | — |
| | | EC N-100 | 4.16 | 20.16 | 4.16 | 20.16 | 3.12 | 15.12 |
| | | Klucel EF | 3.75 | 18.17 | 3.75 | 18.17 | 3.75 | 18.17 |
| | | TEC | 0.60 | 2.91 | 0.60 | 2.91 | 0.6 | 2.91 |
| | | Talc | 10.05 | 48.69 | 10.05 | 48.69 | 10.05 | 48.69 |
| | | Subtotal | 20.7 | 100.0 | 20.7 | 100.0 | 20.7 | 100.0 |

| | | | |
|---|---|---|---|
| Factor/Polymer | EC N-100/ | EC N-100/ | EC N-100/ |
| ratio | Eudragit ® | Eudragit ® | Eudragit ® |
| L100-55 = 2/1 | L100 = 2/1 | L100 = 1/1 | |

FIGS. 9(A) and 9(B) show that increasing the ratio of the pH-dependent polymer (e.g., Eudragit® L100) slowed the dissolution rate and better controlled the release of methylphenidate, and thus the therapy period of the drug can be extended.

The above examples show that the controlled release formulation of the present invention may decrease the number of doses of a drug that need to be administered over time and provide a better balance of desired and undesired pharmacological effects of the drug by controlling the drug release at a suitable rate.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

1. A controlled release formulation comprising:
(A) an outer compressed coat layer comprising
(i) an immediate release granule comprising a first active ingredient and a pharmaceutically acceptable excipient; and
(ii) a controlled release granule comprising a second active ingredient and a controlled release agent; and
(B) an inter layer comprising
(i) a core tablet comprising a third active ingredient and a controlled release agent; and
(ii) an optional controlled release film coating the core tablet.

2. The controlled release formulation as claimed in claim 1, wherein the first, second and third active ingredients are the same.

3. The controlled release formulation as claimed in claim 1, wherein the first, second and third active ingredients are different from each other.

4. The controlled release formulation as claimed in claim 1, wherein the controlled release agent is selected from the following group consisting of hydrophilic polymers, water-swellable polymers, water-insoluble polymers, pH-dependent polymers, hydrophobic materials, or any combination thereof.

5. The controlled release formulation as claimed in claim 1, wherein the controlled release film comprises a film-forming polymer.

6. The controlled release formulation as claimed in claim 1, wherein the first, second and third active ingredients are methylphenidate salt.

7. The controlled release formulation as claimed in claim 6, wherein the controlled release formulation exhibits a ratio of a geometric mean of logarithmic transformed AUC_{0-∞}, of the controlled release formulation to a geometric mean of logarithmic transformed AUC_{0-∞}, of the reference drug (Concerta®) of about 0.80 to about 1.20; a ratio of a geometric mean of logarithmic transformed AUC₀₋ₜ of the controlled release formulation to a geometric mean of logarithmic transformed AUC₀₋ₜ of the reference drug (Concerta®) of about 0.80 to about 1.20; a ratio of a geometric mean of logarithmic transformed pAUC_{0-T1} of the controlled release formulation to a geometric mean of logarithmic transformed pAUC_{0-T1} of the reference drug (Concerta®) of about 0.80 to about 1.20; a ratio of a geometric mean of logarithmic transformed pAUC_{T1-T2} of the controlled release formulation to a geometric mean of logarithmic transformed pAUC_{T1-T2} of the reference drug (Concerta®) of about 0.80 to about 1.20; a ratio of a geometric mean of logarithmic transformed pAUC_{T2-T3} of the controlled release formulation to a geometric mean of logarithmic transformed pAUC_{T2-T3} of the reference drug (Concerta®) of about 0.80 to about 1.20; or a ratio of a geometric mean of logarithmic transformed Cₘₐₓ of the controlled release formulation to a geometric mean of logarithmic transformed Cₘₐₓ of the reference drug (Concerta®) of about 0.80 to about 1.20.

8. The controlled release formulation as claimed in claim 6, wherein the controlled release formulation exhibits at least one of the following pharmacokinetic parameters: (i) a maximum plasma concentration Cₘₐₓ of methylphenidate of about 7 to 30 ng/ml; (ii) an area under the concentration time curve AUC₀₋ₜ or AUC_{0-∞} of methylphenidate of about 130 to 220 ng·hr/ml; and (iii) a first peak plasma Tₘₐₓ₁ of about 1 hour, and/or a second peak plasma Tₘₐₓ₂ of about 6.5 hours under a fasted or a fed condition after oral administration to a patient.

9. A controlled release formulation comprising:
(A) an outer compressed coat layer comprising
(i) an immediate release granule comprising about 0.5% to about 20.0% by weight of a first active ingredient on the basis of the total weight of the immediate release granule, and a pharmaceutically acceptable excipient; and
(ii) a controlled release granule comprising about 0.5% to about 30.0% by weight of a second active ingredient and about 0.5% to about 95.0% by weight of a controlled release agent, on the basis of the total weight of the controlled release granule; and
(B) an inter layer comprising
(i) a core tablet comprising about 20.0% to about 50.0% by weight of a third active ingredient and about 10.0% to about 50.0% by weight of a controlled release agent, on the basis of the total weight of the core tablet; and
(ii) an optional controlled release film coating the core tablet, comprising about 20.0% to about 99.5% by weight of a film-forming polymer, on the basis of the total weight of the controlled release film.

10. The controlled release formulation as claimed in claim 9, wherein the immediate release granule comprises about 3.0% to about 15.0% by weight of the first active ingredient; the controlled release granule comprises about 5.0% to about 20.0% by weight of the second active ingredient and about 30.0% to about 90.0% by weight of the controlled release agent; the core tablet comprises about 30.0% to about 40.0% by weight of the third active ingredient and about 15.0% to about 40.0% by weight of the controlled release agent; and the controlled release film comprises about 30.0% to about 95.0% by weight of the film-forming polymer.

11. The controlled release formulation as claimed in claim 9, wherein the weight ratio of the immediate release granule to the controlled release granule in the outer compressed coat layer ranges from 10/1 to 1/4.

12. The controlled release formulation as claimed in claim 9, wherein the controlled release agent is selected from the following group consisting of hydrophilic polymers, water-swellable polymers, water-insoluble polymers, pH-dependent polymers, hydrophobic materials, or any combination thereof.

13. The controlled release formulation as claimed in claim 9, wherein the first, second and third active ingredients are methylphenidate salt.

14. The controlled release formulation as claimed in claim 13, wherein the controlled release formulation exhibits a ratio of a geometric mean of logarithmic transformed AUC_{0-∞} of the controlled release formulation to a geometric mean of logarithmic transformed AUC_{0-∞}, of the reference drug (Concerta®) of about 0.80 to about 1.20; a ratio of a geometric mean of logarithmic transformed AUC₀₋ₜ of the controlled release formulation to a geometric mean of logarithmic transformed AUC₀₋ₜ of the reference drug (Concerta®) of about 0.80 to about 1.20; a ratio of a geometric mean of logarithmic transformed pAUC_{0-T1} of the controlled release formulation to a geometric mean of logarithmic transformed pAUC₀₄₁ of the reference drug (Concerta®) of about 0.80 to about 1.20; a ratio of a geometric mean of logarithmic transformed pAUC_{T1-T2} of the controlled release formulation to a geometric mean of logarithmic transformed pAUC_{T1-T2} of the reference drug (Concerta®) of about 0.80 to about 1.20; a ratio of a geometric mean of logarithmic transformed pAUC_{T2-T3} of the controlled release formulation to a geometric mean of logarithmic transformed pAUC_{T2-T3} of the reference drug (Concerta®) of about 0.80 to about 1.20; or a ratio of a geometric mean of logarithmic transformed Cₘₐₓ of the controlled release formulation to a geometric mean of logarithmic transformed Cₘₐₓ of the reference drug (Concerta®) of about 0.80 to about 1.20.

15. The controlled release formulation as claimed in claim 13, wherein the controlled release formulation exhibits at least one of the following pharmacokinetic parameters: (i) a maximum plasma concentration Cₘₐₓ of methylphenidate of about 7 to 30 ng/ml; (ii) an area under the concentration time curve AUC₀₋ₜ or AUC_{0-∞} of methylphenidate of about 130 to 220 ng·hr/ml; and (iii) a first peak plasma Tₘₐₓ₁ of about 1 hour, and/or a second peak plasma Tₘₐₓ₂ of about 6.5 hours under a fasting or a fed condition after oral administration to a patient.

16. The controlled release formulation as claimed in claim 13, wherein the formulation has an in vitro dissolution rate when measured by the USP Apparatus II (Paddle) at 50 rpm in 500 ml pH 6.8 PBS at 37° C. and using UV detection at 270 nm, between 10% and 30% methylphenidate released after 1 hour; between 25% and 55% methylphenidate released after 2 hours; between 55% and 85% methylphenidate released after 4 hours; and not less than 90% methylphenidate released after 12 hours, by weight.

17. A controlled release formulation comprising:
(A) an outer compressed coat layer comprising
(i) an immediate release granule comprising about 0.5% to about 20.0% by weight of a first active ingredient on the basis of the total weight of the immediate release granule, and a pharmaceutically acceptable excipient; and
(ii) a controlled release granule comprising about 0.5% to about 30.0% by weight of a second active ingredient and about 0.5% to about 95.0% by weight of a controlled release agent, on the basis of the total weight of the controlled release granule; and
(B) an inter layer comprising
(i) a core tablet comprising about 20.0% to about 50.0% by weight of a third active ingredient and about 10.0% to about 50.0% by weight of a controlled release agent, on the basis of the total weight of the core tablet; and
(ii) an optional controlled release film coating the core tablet, comprising about 20.0% to about 99.5% by weight of a film-forming polymer, on the basis of the total weight of the controlled release film;
wherein the formulation has an in vitro dissolution rate when measured by the USP Apparatus II (Paddle) at 50 rpm in 500 ml pH 6.8 PBS at 37° C. and using UV detection at 270 nm, between 10 and 30% methylphenidate released after 1 hour; between 25% and 55% methylphenidate released after 2 hours; between 55 and 85% methylphenidate released after 4 hours; and not less than 90% methylphenidate released after 12 hours, by weight.

18. The controlled release formulation as claimed in claim 17, wherein the formulation has an in vitro dissolution rate when measured by the USP Apparatus II (Paddle) at 50 rpm in 500 ml 0.1N hydrochloric acid at 37° C. and using UV detection at 270 nm, between 10 and 30% methylphenidate released after 1 hour; between 20 and 50% methylphenidate released after 2 hours; between 35 and 60% methylphenidate released after 4 hours; and between 65 and 90% methylphenidate released after 8 hours, by weight.

19. (canceled)

20. A method for treating Attention-Deficit Disorder (ADD) or Attention-Deficit Hyperactivity Disorder (ADHD) in a patient, comprising administering the controlled release formulation as claimed in claim 1 to said patient.
